(19) United States
(12) Patent Application Publication
(10) Pub. No.: US 2014/0160261 A1
(43) Pub. Date: Jun. 12, 2014

(54) VISUALIZATION INSTRUMENT

(71) Applicant: KING SYSTEMS CORPORATION, Noblesville, IN (US)

(72) Inventors: DAVID J. MILLER, PITTSBURG, KS (US); YUN SIUNG TONY YEH, Libertyville, IL (US); THOMAS W. MCGRAIL, Cicero, IN (US); AMY L. HRUSKA, Indianapolis, IN (US)

(21) Appl. No.: 13/941,183

(22) Filed: Jul. 12, 2013

Related U.S. Application Data
(63) Continuation of application No. PCT/US12/21122, filed on Jan. 12, 2012.
(60) Provisional application No. 61/431,939, filed on Jan. 12, 2011.

(51) Int. Cl. A61B 1/313

(52) U.S. Cl. CPC A61B 1/313
USPC 348/77

(57) ABSTRACT

A visualization instrument comprising a camera assembly and a wireless arrangement, the camera assembly providing digital images to a display device.

### CROSS-REFERENCE TO RELATED APPLICATIONS

This application is a continuation of International Application PCT/US12/21122, with an international filing date of Jan. 12, 2012, which claims the benefit of priority from U.S. Provisional Patent Application Ser. No. 61/431,939 entitled VISUALIZATION INSTRUMENT filed Jan. 12, 2011; the disclosures of said applications are expressly incorporated by reference herein in their entirety.

### FIELD OF THE DISCLOSURE

The present disclosure relates generally to a system including a visualization instrument comprising a camera to view an internal space and, more particularly, to a visualization instrument comprising a camera to examine the interior of a patient.

### BACKGROUND OF THE DISCLOSURE

Visualization instruments include endoscopes, laryngoscopes, borescopes and other medical instruments designed to look inside the body of a patient. Medical visualization instruments are used in a multitude of medical procedures including laryngoscopy, rhinoscopy, bronchoscopy, cystoscopy, hysteroscopy, laparoscopy, arthroscopy, etc. Visualization instruments are also used in non-medical applications such as to investigate the internal structures of machines, buildings, and explosive devices. Laryngoscopes are used to obtain views of the vocal folds and the glottis to perform noninvasive tracheal intubations. A conventional rigid laryngoscope consists of a handle with a light source and a blade. Direct laryngoscopy is usually carried out with the patient lying on his or her back. The laryngoscope is inserted into the mouth, typically on the right side, and pushed towards the left side to move the tongue out of the line of sight and to create a pathway for insertion of an endotracheal tube. The blade may be lifted with an upward and forward motion to move the epiglottis and make a view of the glottis possible. Once the laryngoscope is in place, the endotracheal tube may be inserted into the pathway. The blade may be provided with guide surfaces to guide the insertion of the endotracheal tube. Laryngoscopes may be outfitted with illumination devices and optical devices to provide views of the vocal cords externally of the patient's body. Optical devices include lenses, mirrors, prisms and fiberoptic fibers, all adapted to transfer an optical image. Imaging devices may also be provided to capture the optical images and display the optical images in high definition display monitors. Stylets and other visualization instruments have also been developed.

Traditional visualization instruments have limitations such as, for example, fogging, insufficient lighting to produce a good optical image, inability to project images remotely, additional procedural steps to insert the endotracheal tube, and cost, to name a few.

### SUMMARY OF THE DISCLOSURE

A visualization instrument and a method of using the visualization instrument are disclosed herein. In an exemplary embodiment, the visualization instrument is a video laryngoscope. In another exemplary embodiment, the visualization instrument is configured for non-medical uses. In a further exemplary embodiment, the visualization instrument comprises a display portion including a housing and a display device affixed to the housing; a limited use blade having a proximal end and a distal end, the proximal end forming a handle with a cavity therein configured to removably receive the housing; a camera assembly supported by the distal end of the blade and providing digital images to the display portion; and a wireless arrangement between the display portion and the camera assembly for transferring at least one of power and digital images.

In a further exemplary embodiment, the visualization instrument comprises a display portion including a housing and a display device affixed to the housing; a limited use blade having a proximal end and a distal end, the proximal end forming a handle with a cavity therein configured to removably receive the housing; a camera assembly supported by the distal end of the blade and providing digital images to the display portion; and a wireless arrangement between the display portion and the camera assembly for transferring at least one of power and digital images, wherein the wireless arrangement comprises a wireless transmitter and a wireless receiver coupled, respectively, to the camera assembly and the display portion, the wireless transmitter transmitting digital images from the camera assembly to the wireless receiver.

In a yet further exemplary embodiment, the visualization instrument comprises a display portion including a housing and a display device affixed to the housing; a limited use blade having a proximal end and a distal end, the proximal end forming a handle with a cavity therein configured to removably receive the housing; a camera assembly supported by the distal end of the blade and providing digital images to the display portion; and a wireless arrangement between the display portion and the camera assembly for transferring at least one of power and digital images, wherein the wireless arrangement comprises an inductive coupling having a primary portion coupled to the display portion and a secondary portion coupled to the camera assembly.

In a still further exemplary embodiment, the visualization instrument comprises a display portion including a housing and a display device affixed to the housing; a limited use blade having a proximal end and a distal end, the proximal end forming a handle with a cavity therein configured to removably receive the housing; a camera assembly supported by the distal end of the blade and providing digital images to the display portion; and a wireless arrangement between the display portion and the camera assembly for transferring at least one of power and digital images, wherein the wireless arrangement comprises a wireless transmitter and a wireless receiver coupled, respectively, to the camera assembly and the display portion, the wireless transmitter transmitting digital images from the camera assembly to the wireless receiver, and further comprising an inductive coupling having a primary portion coupled to the display portion and a secondary portion coupled to the camera assembly, wherein the primary portion inductively transmits energy to the secondary portion to power the camera assembly.

In another exemplary embodiment, the visualization instrument comprises a display portion including a housing and a display device affixed to the housing; a limited use blade having a proximal end and a distal end, the proximal end forming a handle with a cavity therein configured to removably receive the housing; a camera assembly supported by the distal end of the blade and providing digital images to the display portion; a wireless arrangement between the display portion and the camera assembly for transferring at least one of power and digital images; and wires having first ends spaced apart from second ends, the first ends coupled to the display portion, wherein the wireless arrangement is formed by an inductive coupling having a primary portion and a secondary portion, the primary portion coupled to the second ends of the wires and the secondary portion coupled to the camera assembly.

In a further exemplary embodiment, the visualization instrument comprises a display portion including a housing and a display device affixed to the housing; a limited use blade having a proximal end and a distal end, the proximal end forming a handle with a cavity therein configured to removably receive the housing; a camera assembly supported by the distal end of the blade and providing digital images to the display portion; and a wireless arrangement between the display portion and the camera assembly for transferring at least one of power and digital images; the camera assembly further comprising a lens, a light guide, and a light source, the light source generating a light, the camera positioned between the light source and the lens, and the light guide guiding the light to a space distal of the lens.

In a still further exemplary embodiment, the visualization instrument comprises a display portion including a housing and a display device affixed to the housing; a limited use blade having a proximal end and a distal end, the proximal end forming a handle with a cavity therein configured to removably receive the housing; a camera assembly supported by the distal end of the blade and providing digital images to the display portion; a wireless arrangement between the display portion and the camera assembly for transferring at least one of power and digital images; and an orientation device for configuring the orientation of the visualization instrument.

In a yet further exemplary embodiment, the visualization instrument comprises a display portion including a housing and a display device affixed to the housing; a limited use blade having a proximal end and a distal end, the proximal end forming a handle with a cavity therein configured to removably receive the housing; a camera assembly supported by the distal end of the blade and providing digital images to the display portion; a wireless arrangement between the display portion and the camera assembly for transferring at least one of power and digital images; and at least one of a temperature sensor and a humidity sensor.

The features of this invention, and the manner of attaining them, will become more apparent and the invention itself will be better understood by reference to the following description of embodiments of the invention taken in conjunction with the accompanying drawings.

### BRIEF DESCRIPTION OF THE DRAWINGS

FIGS. 1 and 2 are perspective views of exemplary visualization instrument embodiments according to the disclosure;

FIGS. 3 and 4 are plan views of the visualization instrument of FIG. 1;

FIG. 5 is a plan view of another embodiment of a blade usable with the visualization instrument of FIG. 1;

FIGS. 6, 7 and 8 are perspective and exploded views of an exemplary embodiment of a camera assembly;

FIG. 9 is a block diagram of electronic circuits corresponding to the visualization instrument of FIG. 1;

FIGS. 10, 11 and 12 are perspective views of additional exemplary embodiments of visualization instruments;

FIG. 13 is an exploded view of another exemplary embodiment of a camera assembly; and

FIG. 14 is a perspective view of yet another exemplary embodiment of a visualization instrument.

Corresponding reference characters indicate corresponding parts throughout the several views. Although the drawings represent embodiments of the present invention, the drawings are not necessarily to scale and certain features may be exaggerated to better illustrate and explain the embodiments. The exemplifications set out herein illustrate embodiments of the invention in several forms and such exemplification is not to be construed as limiting the scope of the invention in any manner.

### DETAILED DESCRIPTION

The embodiments of the disclosure discussed below are not intended to be exhaustive or limit the invention to the precise forms disclosed in the following detailed description. Rather, the embodiments are chosen and described so that others skilled in the art may utilize their teachings.

A visualization instrument and a method of using the visualization instrument are disclosed herein. The visualization instrument is insertable into a space to capture images representing internal views of the space. Additional features of the visualization instrument are disclosed in U.S. patent application Ser. No. 12/874,035, entitled VISUALIZATION INSTRUMENT filed on Sep. 1, 2010, which is expressly incorporated by reference herein in its entirety. Advantageously, embodiments of components of visualization instruments according with the disclosure are configured to be produced at low cost.

While the embodiments of the disclosure are applicable in medical and non-medical applications, exemplary features of visualization instruments will be described below with reference to medical instruments such as laryngoscopes and stylets although the invention is not limited to medical applications and instruments. In some embodiments, visualization instruments include a display device. In other embodiments, the visualization instruments are not attached to a display device and, instead, are adapted to transmit images to another device.

In one exemplary embodiment, a visualization instrument is an intubation device comprising a blade. The blade has a passageway or guide pathway configured to guide insertion of an elongate tubular component. Exemplary elongate tubular components include an airway device, an endotracheal tube and the like. A camera in a camera assembly captures images of the patient being intubated, which are shown with a display device. A distal end of the tubular component may also be visible in the images as the tubular component slides through the guide pathway towards the vocal cords. Exemplary embodiments of blades and intubation devices are disclosed further below which facilitate reusability of portions of the intubation device to reduce intubation costs. In some embodiments, the laryngoscope blade is configured to be discarded after a single use. In other embodiments, the laryngoscope blade is configured to be discarded after a limited number of uses. Features to control limited uses are further described in U.S. patent application Ser. No. 12/874,035, entitled VISUALIZATION INSTRUMENT.

In some exemplary embodiments, a plurality of wires establish a data pathway, a power pathway, or both data and power pathways between the camera and the video device through the blade. The plurality of wires may be referred to as a wire bundle. In one variation, the wire bundle comprises a wire assembly or conductor assembly. The wire bundle is removed from the blade and reused while the blade is discarded. In one example, the wire bundle is removably attached to the display portion. The display portion is reusable while the wire bundle is capable of a limited number of uses.

In one exemplary embodiment, the wire bundle is inductively coupled to the display portion, which is reusable. The wire bundle is attached to the camera assembly and insertable through a blade opening such that the inductive coupling end passes through a blade cavity until it comes near an inductive coupling end of the display portion, thus enabling power to be transferred inductively therefrom. After use, the wire bundle and camera assembly are removed and the blade is discarded.

In another exemplary embodiment, the wire bundle is inductively coupled to the camera assembly and permanently attached to the display portion. The wire bundle is inserted through the blade cavity as the blade is assembled with the display portion, thus making a primary inductive coupling portion attached to the wire bundle available at the distal end of the blade. A secondary inductive coupling portion is attached to the camera assembly. The camera assembly is then coupled to the blade and an electronic pathway is established through the inductive coupling. In one variation, the data pathway is wireless. In another variation, the camera assembly is mechanically and removably attached to the wire bundle.

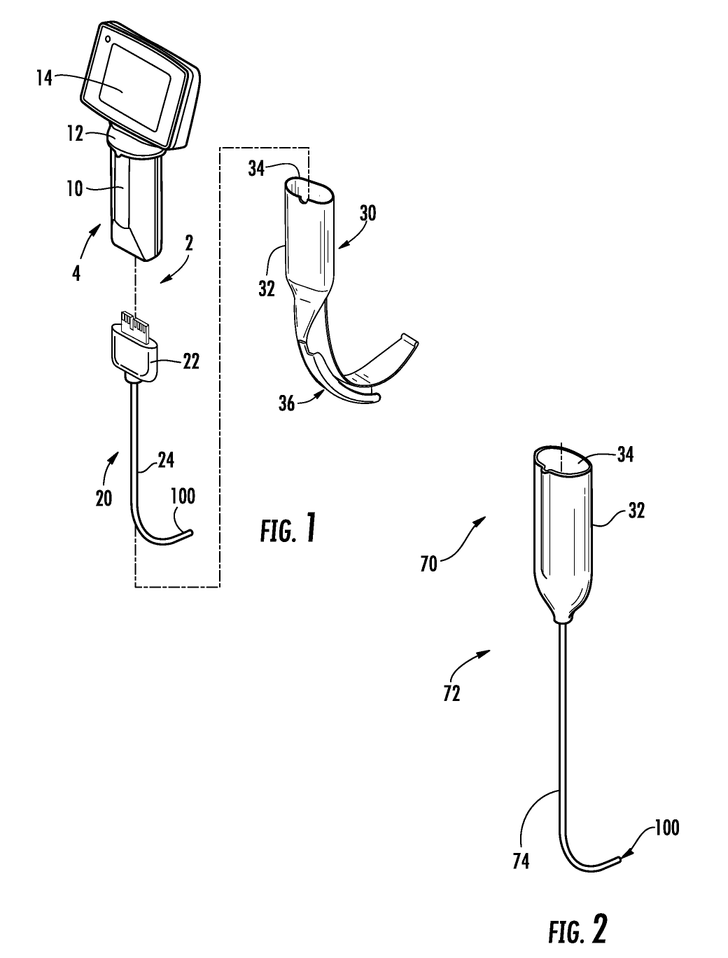

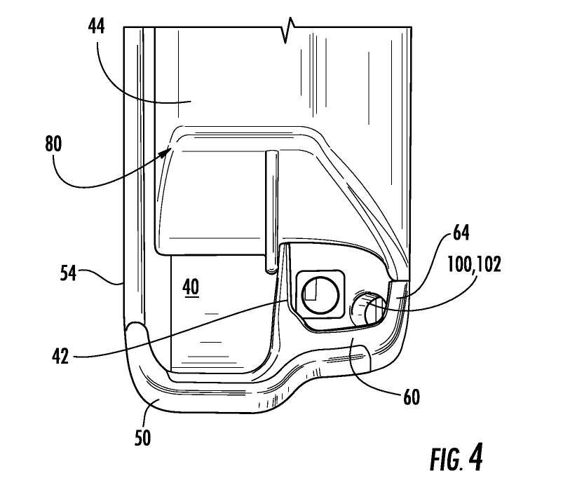

Referring to FIG. 1, an exemplary embodiment of a visualization instrument according to the disclosure is provided. The visualization instrument comprises an intubation instrument 2 including a display portion 4, a wire harness 20, and a blade 30. Display portion 4 has a housing 10, a display device 14, and a support 12 coupling display device 14 and housing 10. Wire harness 20 includes a connector 22 electrically coupled to a plurality of conductors 24. A camera assembly 100 is connected to conductors 24. Blade 30 comprises a handle 32 in a proximal end thereof spaced apart from an insertable portion 36 located at a distal end. Camera assembly 100 is located at the distal end of insertable portion 36 when wire harness 20 is assembled with blade 30. In the present embodiment, blade 30 is shown as a single part integrally combining handle 32 and insertable portion 36. In a variation thereof, the handle and the insertable portion are distinct parts that are removably attachable. Handle 32 comprises a proximal cavity 34 receiving housing 10 and coupling display portion 4 to blade 30. Insertable portion 36 comprises an elongate passageway, illustratively passageway 40 (shown in FIGS. 3 and 4), designed to guide insertion of a catheter, intubation tube and the like (not shown) into the larynx of a patient. In the present embodiment, camera 100 is inserted through proximal cavity 34 after connector 22 is electrically coupled to display portion 4. Blade 30 slides over conductors 24 until proximal cavity 34 fully receives housing 10 to complete assembly of intubation instrument 2.

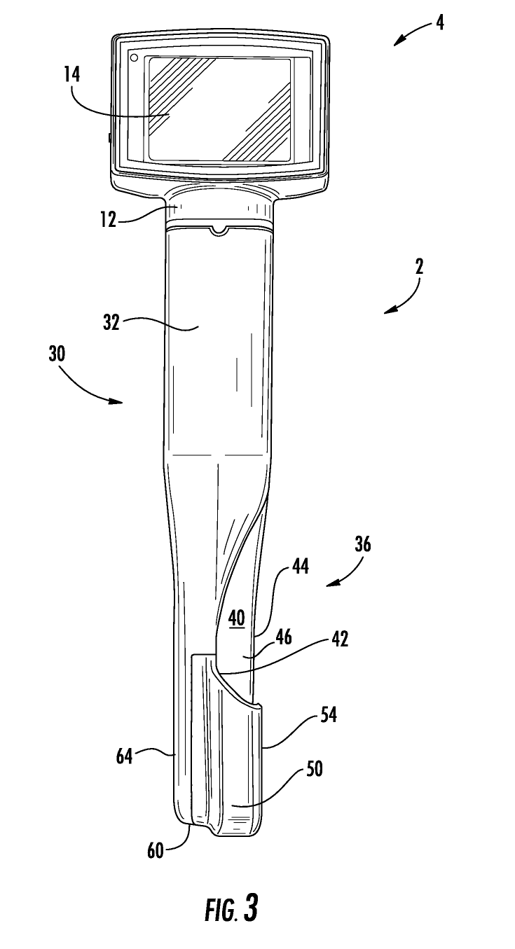

Referring to FIG. 2, another exemplary embodiment of a visualization instrument according to the disclosure is provided. The visualization instrument comprises display portion 4, wire harness 20, and a blade 70. Blade 70 comprises a handle 32 in a proximal end thereof spaced apart from an elongate portion 72 located at a distal end. Elongate portion 72 has a tubular arm 74 receiving conductors 24 and camera assembly 100 therein. Assembly and operation of the present visualization instrument is similar to that of intubation instrument 2. A visualization instrument as in the present embodiment can be used in medical, industrial, and other non-medical applications. In one example, elongate portion 72 comprises a stylet.

Referring to FIGS. 3 and 4, additional details of blade 30 are disclosed. The distal end of blade 30 comprises a guide pathway 40 on one side of a medial wall 42 and an electronics pathway (not shown) on the opposite side of medial wall 42. As shown, blade 30 further comprises anterior guide wall 44 having an anterior guide surface 46. The distal portion of guide pathway 40 is defined by medial wall 42, a posterior guide wall 50, a lateral guide wall 54, and anterior wall 44. The electronics pathway is defined by medial wall 42, a posterior electronics pathway wall 60, a lateral electronics pathway wall 64, and anterior wall 44. In the present embodiment, conductors 24 are located in the electronics pathway when intubation instrument 2 is assembled. As best seen in FIG. 4, a distal cavity, denoted by numeral 102, receives therein camera assembly 100. A tip portion 80 of blade 30 extends distally beyond the electronics pathway. In another variation of the present embodiment, the guide pathway is located on the posterior side of the blade.

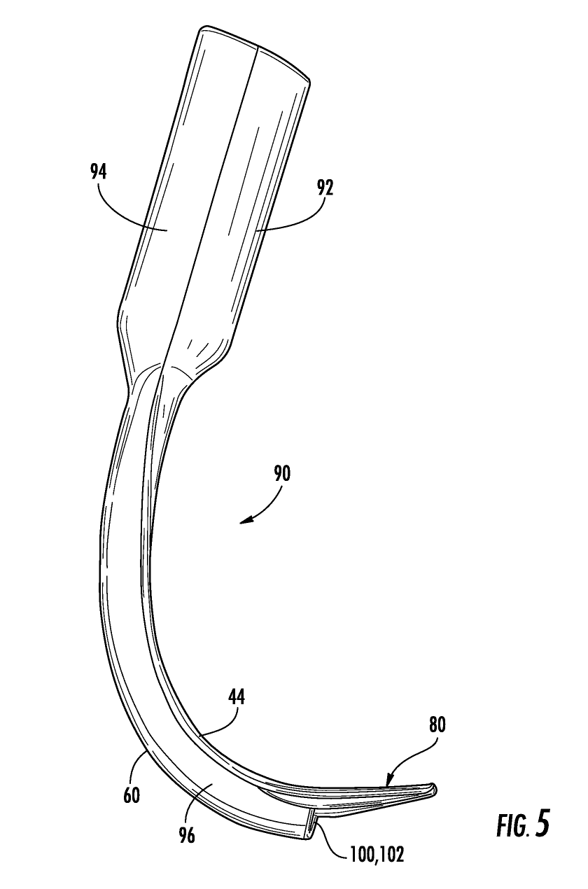

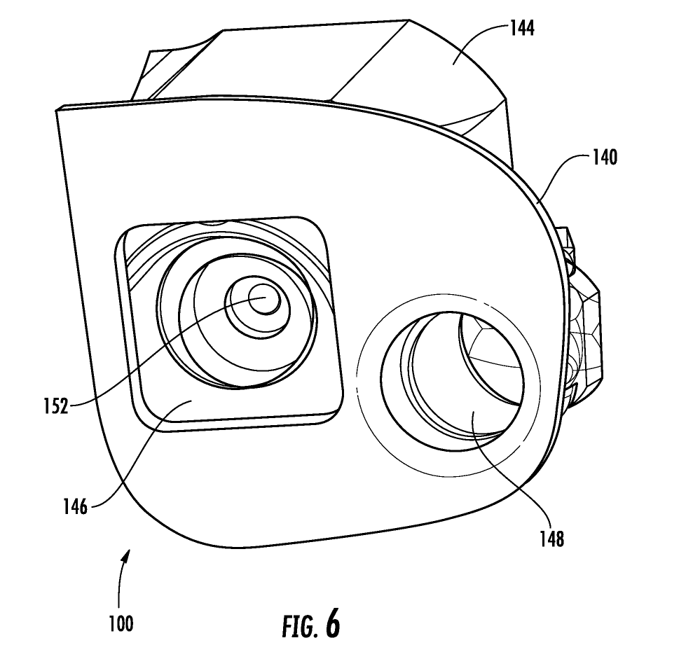

An exemplary embodiment of a blade 90 without posterior and lateral guide walls is illustrated in FIG. 5. The anterior and posterior sides of blade 90 are denoted by numerals 92 and 94. A medial wall is denoted by numeral 96. Medial wall 96 is substantially the same as medial wall 42. Some surgeons may prefer the additional freedom to control the endotracheal tube (without the lateral and posterior guide walls) provided by blade 90 as compared to blade 30. Except for the guide pathway differences, blade 90 and blade 30 are interchangeable.

In a further exemplary embodiment, the blade is assembled without glue. In one example, the blade comprises two portions which are ultrasonically bonded. In another example, the two portions are mechanically fastened. Additional features are provided to enable soaking of the blade. In one example of the present embodiment, a molded cover is provided which is attached to the blade to seal the electrical contacts. In another example of the present embodiment, a disposable plastic sheath is provided which covers the blade during use. In a further example of the present embodiment, the blade comprises a stylet and the sheath covers the stylet. In a yet further example of the present embodiment, the optical components of the imaging assembly are potted in the blade.

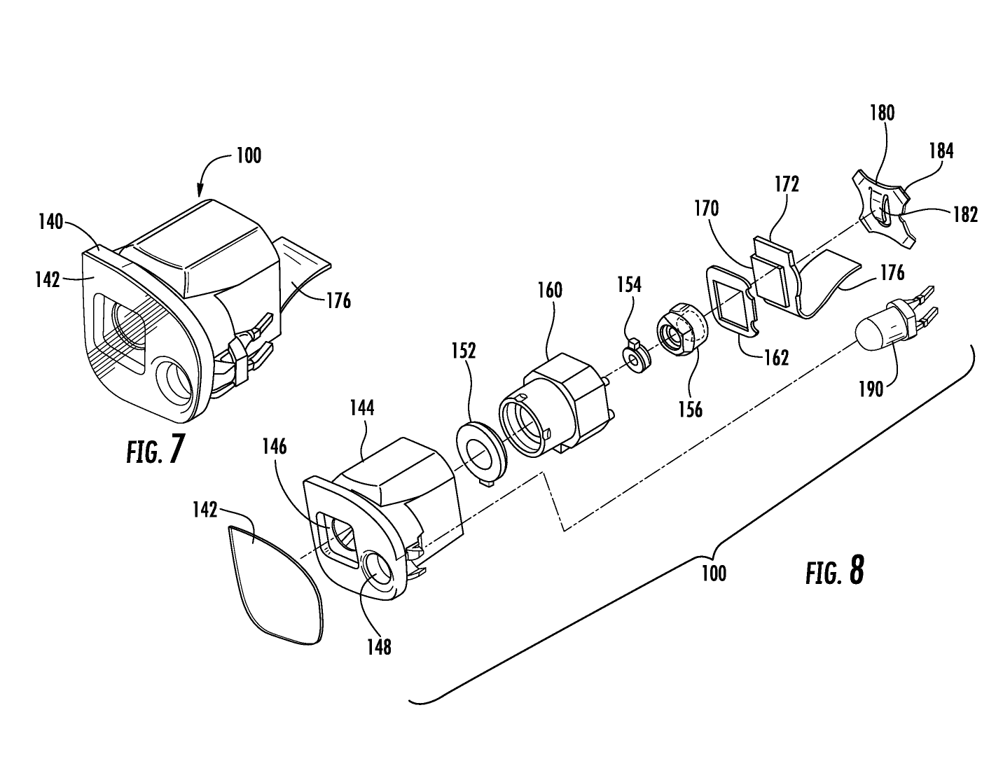

FIGS. 7, 8 and 9 are perspective and exploded views of an exemplary embodiment of a camera assembly, illustratively camera assembly 100. Camera assembly 100 comprises a distal cover 142, a camera holder 144 having an imaging opening 146 and an illumination opening 148 disposed on a distal wall 140, a distal lens 152, a camera barrel 160, lenses 154 and 156, a gasket 162, a camera 170, a support board 172 supporting camera 170, a connector assembly 176, and a backing plate 180 having a pressure component 182 and a plurality of locking components 184. Backing plate 180 presses support board 172 and lenses 154 and 156 in camera barrel 160 to retain them in place. Camera barrel 160 is positioned in a cavity (not shown) of camera holder 144 to hold distal lens 152 inside camera holder 144. Gasket 162 is optional and may be removed. If used, gasket 162 seals imaging sensor 170 inside camera barrel 160 and prevents light from entering camera barrel 160 and degrading the images. An illumination source, illustratively LED 190, is coupled to camera holder 144 to illuminate the space in front of distal cover 142 through illumination opening 148. In one variation of the previous example, distal cover 142 is adhesively bonded to camera holder 144 using a silicone release application method. The combination of a support housing and an imaging barrel simplifies assembly of the camera assembly. In a variation of the example described above, only two lenses are used.

In another variation of the present embodiment, the camera barrel and the camera holder are formed by single-mold inserts which cause the distal lens and second lens to self-align relative to the optical axis of the camera assembly which simplifies the assembly process and reduces cost by eliminating the need for focusing features. In yet another variation thereof, the camera assembly comprises a distal lens having a negative meniscus, and a doublet comprised of biconvex and negative meniscus elements. The distal and doublet lenses have aspheric surfaces which, combined with the meniscus and biconvex elements, achieve nearly diffraction limited performance.

A commercially available camera, such as a camera used in cellular phones and personal digital assistants (PDAs), comprises an image sensor and electronic components configured to convert pixel data captured by the image sensor to image data, e.g., digital images, and to output streams of digital images in a standard format. Image sensors may comprise CCD, CMOS sensors with active or passive pixels, or other photo sensors well known in the art. Operational signals are provided to the image sensor to control its operation. Advantageously, the cost of the disposable portion of the visualization instrument is reduced further by locating the components providing the operational signals in the display portion. In one embodiment, a display driver configured to receive the image stream and drive the display device accordingly, also comprises the components necessary to control the camera. In one example thereof, the input/output signals are provided by signal conductors, e.g., a multi-conductor flexible ribbon. In another example thereof, a control component is provided intermediate the camera and the display driver to transform the standard image stream into a differently structured image stream conforming to the size of the display device and/or transforming the standard image stream to a different format corresponding to the format required by the display driver. In a further example thereof, the operational circuits are integrated with the camera, which is configured to output a preconfigured image stream upon the application of power, and which is usable directly by the display device. In yet another example, control components supported by the display portion housing provide control signals to the camera to define the size of the images output by the camera. In a further example, the image stream output by the camera is transmitted wirelessly by a wireless transmitter located in the insertion portion. In yet a further example, the wireless transmitter is integrated with the camera. In a variation thereof, the wireless transmitter is positioned in the proximal end of the insertable portion or in the distal cavity. In one example, the camera forms a digital image stream using radiation having wavelengths ranging between 10 nanometers and 14,000 nanometers. The wavelengths include the visible light, ultraviolet, and infrared spectrums. In one variation, the camera is an infrared camera. In another variation, the camera is an ultraviolet light camera. In another variation, the camera is a visible light camera.

In one exemplary embodiment of a video processing scheme applicable in combination with any of the foregoing or following visualization instrument embodiments, the camera supplies a first image stream which is 8-bits wide. The resolution of the camera is 640×480 (VGA) pixels per frame. There are 30 frames per second. The data format is 2 bytes per pixel (i.e., the so called YUV (4:2:2) format). Intensity Y is specified at every pixel, color information U or V every second time. A FPGA is programmed to convert the data stream to a second image stream with a format compatible with the display device 14 which comprises an OLED display. In an alternative embodiment, the camera data is provided to the video processing chip, and the video processing chip, after adding information such as colors, symbols or other information, outputs a video stream to the FPGA for the FPGA to convert to the VGA format. The display resolution is 320×240 (QVGA) pixels per frame, 30 frames per second. The data format, however, is RGB (6, 6, 6). This format uses a 6-bit value for red, a 6-bit value for green, and a 6-bit value for blue. There are specific well known equations for conversion from the YUV color space to the RGB color space. The FPGA implements this conversion. It also performs the conversion (e.g. dropping every second pixel) to convert from VGA to QVGA resolution. The FPGA also provides signals for writing the converted data stream into the OLED display's memory/buffer. The FPGA also sends the camera data to the NTSC/S-video conversion chip. The video chip having the video processor is capable of accepting the VGA, YUV format almost directly. The FPGA provides the necessary operational signals to load the video chip's memory. In a variation thereof, the FPGA also verifies the identity of the camera against a database of approved cameras. The FPGA extracts camera information from the camera, for example a built-in camera ID or a programmable camera ID, and checks the identity against an approved list which is periodically updated. If the camera identification is not on the approved list, the FPGA does not convert the first image stream or, optionally, inserts a warning into the second image stream to alert a practitioner that the insertable portion is not an approved device. Approval may be desirable to ensure the insertable portion meets quality specifications.

In a further exemplary embodiment of a visualization instrument, the visualization instrument comprises the components described in the preceding paragraph except that the FPGA is substituted with a suitable integrated circuit configured to perform the functions described in the preceding paragraph and other functions further described throughout the disclosure. Suitable integrated circuits include digital signal processors. In a variation of the present embodiment, the visualization instrument comprises both FPGA and digital signal processor.

A program and data structures are embedded in the memory. The program comprises a plurality of processing sequences operable by the processor to interact with data structures containing data. Data includes video instructions, security feature instructions, landmark patterns and the like. In a further exemplary embodiment of a display portion suitable for use in any of the visualization instruments described herein, a display portion comprises at least one of temperature and humidity sensors, and data includes at least one of status information and environmental information. Exemplary status information includes battery charge level and number of uses. Exemplary environmental information includes temperature and humidity levels. Such data can be displayed by the display device or transmitted to a remote device to assist the practitioner. Suitable alarm functions can be implemented if the data falls outside a predetermined range. Ranges can be predetermined to alert practitioners to situations where, due to environmental or status conditions, image quality or instrument reliability is negatively and materially affected.

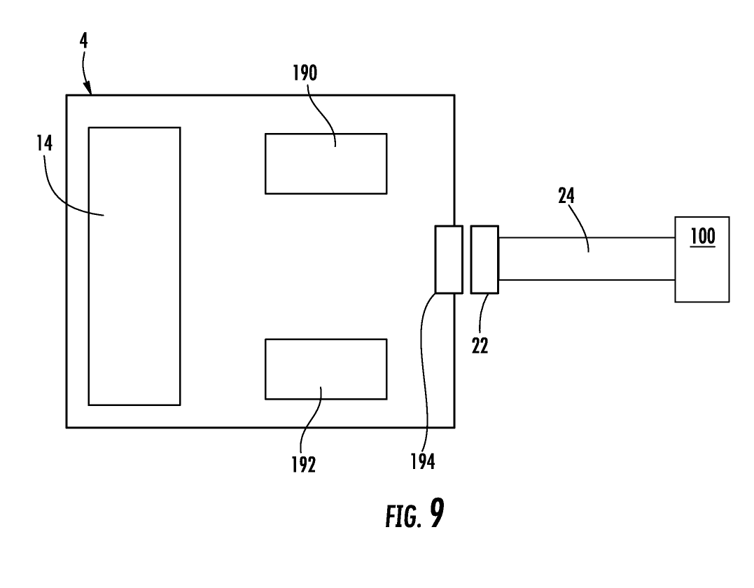

Referring to FIG. 9, a block diagram of electronic circuits corresponding to the visualization instrument of FIG. 1 is shown. The block diagram illustrates display portion 4 comprising display device 14, a power storage device, illustratively batteries 190, video circuits 192, and a connector 194 adapted to electronically couple with connector 22 to thereby place camera assembly 100 in communication with video circuits 192. Batteries 190 power the illumination device and camera 170. Video images are transferred from camera 170 to video circuits 192 through conductors 24, and subsequently, after video processing, to display device 14. In one example suitable for use with the exemplary embodiments described herein, video circuits 192 include memory, an FPGA and/or digital signal processor, and any other components described in the preceding paragraphs.

In one exemplary embodiment, the visualization instrument comprises orientation devices configured to determine the orientation of the visualization instrument. Exemplary orientation devices include inertial devices, such as accelerometers and gyroscopes, and inclinometers. In one example, the visualization instrument utilizes the orientation devices to determine that its position is reversed, and causes video circuits 192 to reverse the video image presented in video display 14. For example, while the instrument is typically used with the anterior side “up”, it may also be used with the anterior side “down”, in which case, according to the present embodiment, the image in the video display is reversed. This feature is useful when the patient is not laying on his/her back.

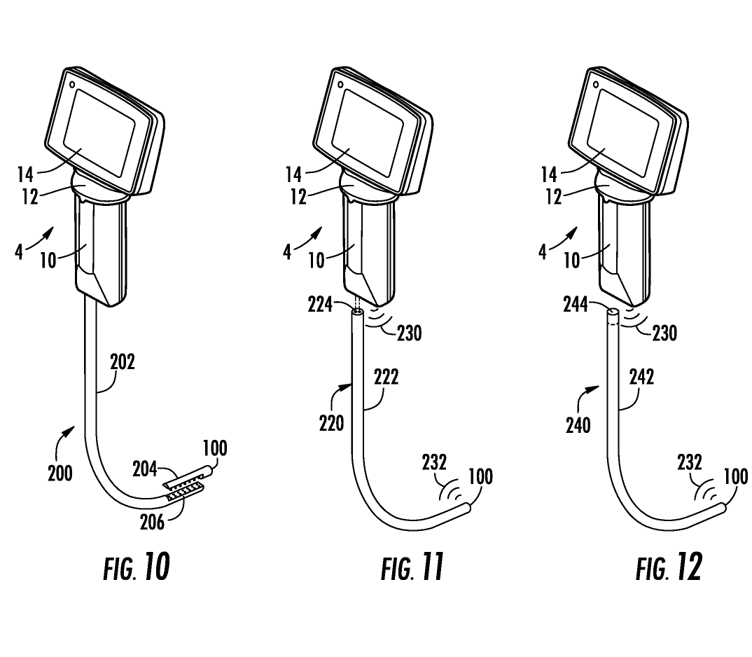

FIGS. 10 to 12 illustrate exemplary embodiments of data and power pathways. As shown in FIG. 10, the visualization device includes hardwired detachable wireless and power pathways. In the present embodiment, display portion 4 is coupled to a wire harness 200 comprising conductors 202 and a connector 206. A connector 204 is adapted to mate with connector 206 and camera assembly 100 thereby permitting separation of camera assembly 100 from wire harness 200. In one variation of the present embodiment, wire harness 200 is affixed to display portion 4. In another variation of the present embodiment, wire harness 200 is removably coupled to display portion 4 and is disposable. Advantageously, removing connector 204 enables camera assembly 100 to be reused with a new wire harness 200 after a first wire harness 200 is discarded after one or a limited number of uses. In a further variation of the present embodiment, the visualization device includes a wireless data pathway and an inductive power pathway.

Connectors 204 and 206 are replaced with an inductive coupling comprising the power pathway and including a primary induction assembly and a secondary induction assembly. The primary induction assembly generates a high frequency oscillating signal which induces a corresponding signal in the secondary induction assembly. In one example, the assemblies are tuned to a resonating frequency which enables inductive transfers at longer gap distances than are possible without resonance. The primary induction assembly is coupled to conductors 202 and includes a primary induction coil adapted to transfer power to a secondary induction coil included with the secondary induction assembly from a power storage device included in the display portion. The primary induction assembly generates a high frequency oscillating signal which induces a corresponding signal in the secondary induction assembly. The secondary induction assembly converts an induced high frequency signal to DC power usable by the camera assembly to illuminate, capture images, and transmit video wirelessly to display portion 4. In one example, the power storage device is a battery located in the housing of the display portion. Wireless transceivers comprise the data pathway. A wireless transceiver is electronically coupled to the imaging assembly and another is electronically coupled to the display portion. Camera assembly 100 also comprises logic to decode wireless transmissions, including data and control information. The wireless transceiver and the camera assembly are inductively powered. In one variation, the wireless transceiver and the camera assembly are integrated and detachable from the blade. The housing of the display portion and connectors 204 are inserted into the blade through the handle while the integrated transceiver/camera assembly is inserted into a cavity in the electronics pathway. In one example, the integrated transceiver/camera assembly are inserted through the distal cavity. In another example, the integrated transceiver/camera assembly is inserted through a side opening (not the distal cavity). An exemplary side opening is shown in FIG. 14 as opening 382. In another example, the camera and transceiver are integrated and removable while the lenses are integrated with the blade. When the camera/transceiver is inserted into the side opening, the camera is aligned with the lenses.

Referring to FIG. 11, an exemplary embodiment of an intubation instrument with a wireless data pathway and a hard-wired power pathway is provided. Transceivers (not shown) are included in display portion 4 and camera assembly 100, corresponding to waves 230, 232, respectively. A wire harness 220 includes power conductors 222 and a connector 224, adapted to be coupled to display portion 4 to transfer power therefrom to camera assembly 100. In one example, wire harness 220 is attached to display portion 4 before both are inserted through the handle of the blade. Camera assembly 100 thus slides into an opening in the distal end of the blade.

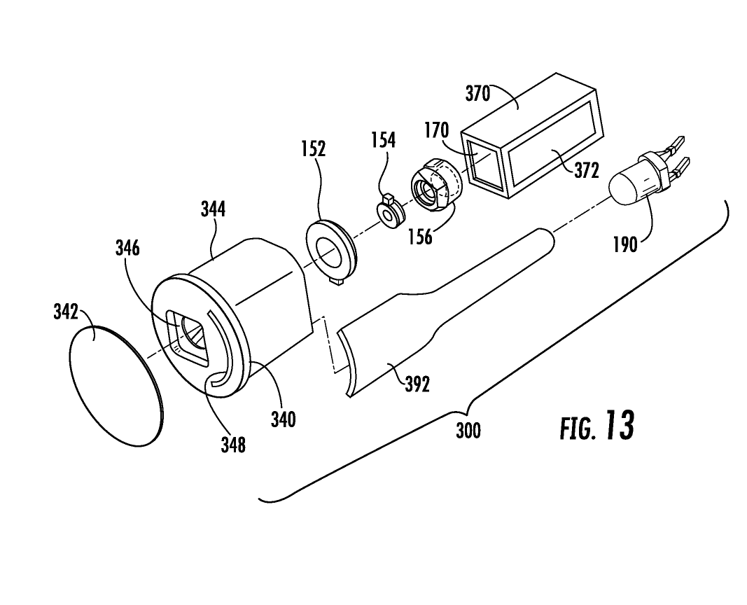

Referring to FIG. 12, an exemplary embodiment of an intubation instrument with a wireless data pathway and an inductive power pathway is provided. Transceivers (not shown) are included in display portion 4 and camera assembly 100, corresponding to waves 230, 232, respectively. In the present embodiment, as in the wireless pathway examples discussed above, camera assembly 100 also comprises logic to decode wireless transmissions, including data and control information. A wire harness 240 includes power conductors 242 and a secondary induction assembly 244, adapted to inductively couple with a primary induction assembly (not shown) supported by display portion 4. The primary induction assembly generates a high frequency oscillating signal which induces a corresponding signal in the secondary induction assembly 244. The secondary induction assembly 244 then converts the high frequency signal to DC power usable by camera assembly 100 to illuminate, capture images, and transmit video wirelessly to display portion 4. In one example, wire harness 240 and camera assembly 100 are inserted through a distal opening of the blade until camera assembly 100 is seated in a distal cavity provided in the electronics pathway for that purpose. Conductors 242 are sufficiently long such that when camera assembly 100 is seated secondary induction assembly 244 is able to inductively couple with the primary induction assembly in display portion 4.

In further exemplary embodiments, the visualization instruments include an actuation mechanism to adjust the distance between camera assembly 100 and the distal end of display portion 4. In one example thereof, the actuation mechanism comprises a biased component which is biased towards the distal end of display portion 4 and adapted to couple the proximal end of the wire harness such that, when camera assembly 100 and the wire harness are inserted into a cavity of the blade, camera assembly 100 bottoms-out on the distal end of the cavity of the blade and pushes the wire harness against the biased component, which retracts away from the distal end of display portion 4. Advantageously, the actuation mechanism permits use of a standard length wire harness with differently sized blades. In one variation of the present embodiment, the actuation mechanism comprises two retraction guides, a biased component and a sliding element adapted to couple the retraction guides and slide therein. The retraction guides are coupled to internal surfaces of the housing of the display portion. An exemplary biased component is a spring. If the wire harness comprises a connector, the sliding element comprises a mating connector adapted to mate with the connector of the wire harness. In one example, the retraction guides comprise slots and the sliding element extends between the slots and slides therein. In another example, the retraction guides comprise elongate ridges and the sliding element comprises, at its opposite ends, slots configured to slide on the ridges. A stop prevents the sliding element from retracting more than a predetermined amount into the housing. In one example, the biased component is coupled between the stop and the sliding element to bias the sliding element towards the distal end of the housing. Application of an external force opposite the bias causes the sliding element to retract. In one example, the predetermined amount is less than 10 mm. In one example, the mating connector is electrically coupled to looped wires which are, at their opposite ends, electrically coupled to the display portion. The loops in the wires enable the mating connector to retract while maintaining an electrical connection between the mating connector and the connector of the wire harness. In another variation of the present embodiment, the actuation mechanism comprising the two retraction guides, the biased component and the sliding element is configured in a cartridge affixed to the wire harness. Retraction occurs within the cartridge.

In one example the biased component includes a connector adapted to couple with connector 224. In another example, the biased component includes a primary induction assembly and is adapted to couple secondary induction assembly 224. In one variation of the present embodiment, the actuation mechanism automatically matches the length of the wire harness to a selected blade or stylet. In one example, a biasing force is provided by a spring to bias the biased component. In another example, the actuation mechanism comprises a motor configured to translate the biased component in response to a measured pressure. In another variation of the present embodiment, the actuation mechanism manually matches the length of the wire harness to a selected blade or stylet. In one example, the actuation mechanism comprises a sliding lever sliding in a slot in the housing with a knob attached to the slide and externally accessible such that a user can actuate the knob to move the slide. Inside the housing, the slide is coupled to the mating connector which mates with the wire harness connector. In another example, a rotatable knob positioned externally of the housing is connected to an axis that extends into the housing. When the knob is rotated, the wire harness retracts or extends. In one example the actuation mechanism includes a gear rotatable by the knob and coupled to a linear gear axially aligned with the display portion and adapted to retract or extend the wire harness.

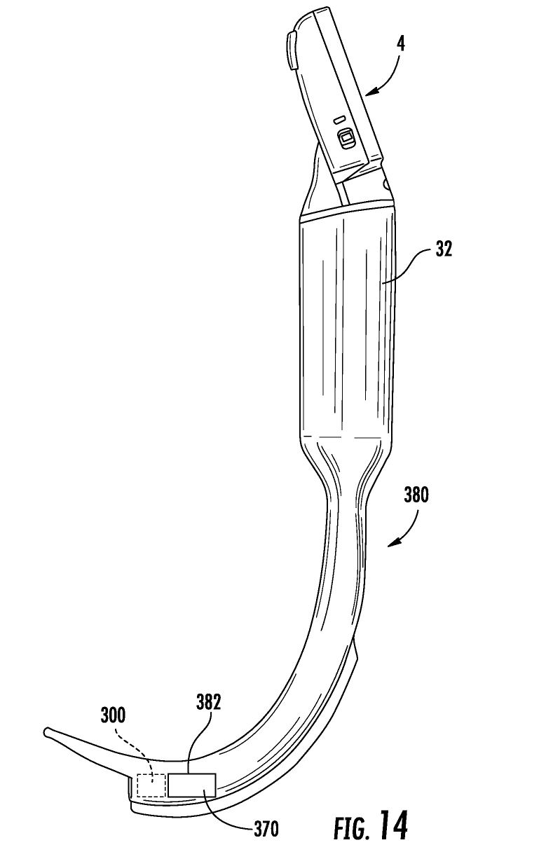

Referring to FIG. 13, an exploded view of another exemplary embodiment of a camera assembly is shown. Camera assembly 300 comprises a distal cover 342, a lens holder 344 having an imaging opening 346 and an illumination opening 348 disposed on a distal wall 340, a distal lens 152, lenses 154 and 156, a camera 170, a light pathway 392, and an illumination source, illustratively LED 190. LED 190 generates light which light pathway 392 transfers to illuminate the space in front of imaging opening 346 through illumination opening 348. Lens holder 344 and one or more of distal lens 152 and lenses 154 and 156 may be referred to as an optical assembly. The optical assembly may also comprise light pathway 392. Advantageously, the light pathway enables the cross-section of the camera assembly to be reduced. In one example, light pathway 392 comprises fibers. Lenses 142, 154 and 156 are enclosed by lens holder 344. Also shown in FIG. 13 is a housing 370 supporting camera 170 and a power storage device. In the example shown, a door 372 is provided to enable insertion and removal of the power storage device. In another example, door 372 is not used and housing 370 is discarded when the power storage device is discharged. In a further example, housing 370 includes contacts (not shown) adapted to receive power, a wireless transceiver, and a secondary induction assembly in addition to camera 170. In the present example, as in variations of the examples provided above, housing 370 is removable from the blade and reusable while the lens holder and lenses are permanently attached to the blade. Advantageously, removability of the camera and other electronic components reduces the cost of disposing of the blade. In another variation, housing 370 supports the illumination source while the optical assembly supports the light pathway. In a further example, housing 370 also supports the optical components and the entire optical/electronic assembly is reusable. A side opening in the electronics pathway of the blade (an exemplary opening is shown in FIG. 14 as opening 382) is provided to receive the battery compartment and camera. In a further variation, the optical assembly is potted or otherwise sealed in the distal cavity of the blade. In a further variation, the removable housing containing the camera is also sealed and adapted to be cleaned with fluids.

Another exemplary embodiment of an intubation instrument is provided in FIG. 14. The instrument includes display portion 4, housing 370, a blade 380, camera assembly 300, and a side opening 382 in the electronics pathway of blade 380. As discussed previously, housing 370 is inserted through side opening 382 and removed from blade 380 after use. In one example, housing 370 includes a camera, a wireless transceiver, and a secondary induction assembly. In another example, housing 370 includes a camera, a wireless transceiver, and a power storage device. In a further example, housing 370 includes a camera, an optical assembly, a wireless transceiver, and a power storage device or a secondary induction assembly. In a yet further example, housing 370 includes a camera, a light guide, a wireless transceiver, and a power storage device or a secondary induction assembly. In a still further example, housing 370 includes a camera, a light source, a wireless transceiver, and a power storage device or a secondary induction assembly.

While the invention has been described as having exemplary designs, the present disclosure may be further modified within the spirit and scope of this disclosure. This application is therefore intended to cover any variations, uses, or adaptations of the disclosure using its general principles. In another variation, the wire bundle is provided in different lengths to suit different blades. In a further variation, a system is provided comprising one camera assembly and multiple blades, which may be the same or different. In another variation, the display portion includes a housing without a display device but including video circuits and a communication component to transfer video images to another display device. Exemplary communication components include a connector and a wireless transceiver. Further, this application is intended to cover such departures from the present disclosure as come within known or customary practice in the art to which this invention pertains.

1. A visualization instrument comprising:
a display portion including a housing and a display device affixed to the housing;
a limited use blade having a proximal end and a distal end, the proximal end forming a handle with a cavity therein configured to removably receive the housing;
a camera assembly supported by the distal end of the blade and providing digital images to the display portion; and
a wireless arrangement between the display portion and the camera assembly for transferring at least one of power and digital images.

2. A visualization instrument as in claim 1, wherein the wireless arrangement comprises a wireless transmitter and a wireless receiver coupled, respectively, to the camera assembly and the display portion, the wireless transmitter transmitting digital images from the camera assembly to the wireless receiver.

3. A visualization instrument as in claim 1, wherein the wireless arrangement comprises a wireless transmitter and a wireless receiver coupled, respectively, to the camera assembly and the display portion, the wireless transmitter transmitting digital images from the camera assembly to the wireless receiver, and further comprising an inductive coupling having a primary portion coupled to the display portion and a secondary portion coupled to the camera assembly, wherein the primary portion inductively transmits energy to the secondary portion to power the camera assembly.

4. A visualization instrument as in claim 1, wherein the blade is configured to removably receive the camera assembly, wherein the camera assembly is not physically attached to the display portion prior to being received by the blade.

5. A visualization instrument as in claim 7, wherein the blade comprises a lateral wall with an opening therein configured to receive the camera assembly therethrough.

6. A visualization instrument as in claim 7, wherein the camera assembly includes an energy storage device.

7. A visualization instrument as in claim 1, further comprising wires having first ends spaced apart from second ends, the first ends coupled to the display portion, wherein the wireless arrangement is formed by an inductive coupling having a primary portion and a secondary portion, the primary portion coupled to the second ends of the wires and the secondary portion coupled to the camera assembly.

8. A visualization instrument as in claim 10, wherein the secondary portion of the inductive coupling is located in the distal end of the blade when the display portion is received by the cavity of the blade.

9. A visualization instrument as in claim 1, further comprising an orientation device for configuring the orientation of the visualization instrument.

10. A visualization instrument as in claim 1, further comprising at least one of a temperature sensor and a humidity sensor.

11. A visualization instrument comprising:
a display portion including a housing and a display device affixed to the housing;
a limited use blade having a proximal end and a distal end, the proximal end forming a handle with a cavity therein configured to removably receive the housing;
a camera assembly supported by the distal end of the blade and providing digital images to the display portion; and
a wireless arrangement between the display portion and the camera assembly for transferring power from the display portion to the camera.

12. A visualization instrument as in claim 11, wherein the wireless arrangement comprises an inductive coupling having a primary portion coupled to the display portion and a secondary portion coupled to the camera assembly.

13. A visualization instrument as in claim 12, wherein the primary portion is configured to inductively transmit energy to the secondary portion to power the camera assembly.

14. A visualization instrument as in claim 11, wherein the blade is configured to removably receive the camera assembly.

15. A visualization instrument as in claim 11, the camera assembly including a lens, a light guide, and a light source, the light source generating a light, the camera positioned between the light source and the lens, and the light guide guiding the light from the light source to a space distal of the lens.

16. A visualization instrument as in claim 15, wherein the light guide is configured to shape the light from the light source in a predetermined pattern.

17. A visualization instrument as in claim 15, wherein the a light guide and the light source are arranged and configured to reduce a cross-sectional area of the camera assembly relative to a second cross-sectional area which the camera assembly comprising the smallest cross-sectional area the camera assembly would exhibit if the camera were not positioned between the light source and the lens.

18. A visualization instrument as in claim 15, wherein the blade is configured to removably receive the camera assembly.

19. A visualization instrument as in claim 15, wherein the light source comprises a light emitting diode.

20. A visualization instrument comprising:
a display portion including a housing and a display device affixed to the housing;
a blade having a proximal end and a distal end, the proximal end forming a handle with a cavity therein configured to removably receive the housing;
a camera assembly supported by the distal end of the blade and including a camera configured to provide digital images to the display portion, the camera assembly including a lens, a light guide, and a light source, the light source generating a light, the camera positioned between the light source and the lens, and the light guide guiding the light from the light to a space distal of the lens.
